(19) United States
(12) Patent Application Publication
(10) Pub. No.: US 2021/0290229 A1
(43) Pub. Date: Sep. 23, 2021

(54) SUTURE INVENTORY MANAGEMENT CART

(71) Applicant: Chase Smith, Nashua, NH (US)

(72) Inventor: Chase Smith, Nashua, NH (US)

(21) Appl. No.: 16/826,355

(22) Filed: Mar. 23, 2020

(51) Int. Cl. A61B 17/06; A61B 50/18; A61B 50/13

(52) U.S. Cl. CPC A61B 17/06133; A61B 50/13; A61B 50/18

(57) ABSTRACT

The present disclosure relates to a suture inventory management device that stores and dispenses individual units of sutures from a first compartment and stores boxes of units of sutures of the same type of sutures in a second compartment. In some embodiments, the suture inventory management device is a suture storage cart comprising suture storage systems. The suture storage systems including at least one pair of suture storage compartments, the pair of suture storage compartments comprising a bulk suture storage compartment and a suture unit storage compartment. Units of sutures are stored in the suture unit storage compartment and boxes of the same type of sutures are stored in the corresponding bulk suture storage compartments. The interior of the suture unit storage compartment has a hinged top lid transparent door that opens by sliding vertically and the bulk suture storage compartments are enclosed by transparent doors.

### FIELD OF DISCLOSURE

The present disclosure relates generally to medical supplies and more specifically to supplying and dispensing sutures. The present disclosure advantageously provides an inventory management device that dispenses individual units of sutures from a first compartment and stores bulk quantities of sutures for future use in a second compartment, where the quantities in both compartments are easily visible, resulting in low quantities in either compartment to be replenished by staff as needed.

### BACKGROUND

The storage of medical supplies in areas in which they are regularly consumed must constantly be monitored and replenished to ensure access to the supplies as they become needed. Each type of bandage, syringe, and implement must be meticulously inventoried on a frequent and routine basis to ensure that patients do not endure undue delay in receiving treatment. Supplies are often kept in various places in a medical office or surgical suite. Medical supplies may be kept on carts, on shelves, in cabinets, and in drawers, and the storage of these supplies may not be uniform from suite to suite. Given the hundreds of medical supplies used to treat patients and the difficulty in constant monitoring of often inadequate storage and inventory protocols of these supplies, time must often be spent locating necessary supplies within a given room and/or locating supplies from a location outside of the area in which treatment is being provided.

Sutures are used in nearly every type of medical practice, from operating rooms, to emergency clinics. Additionally, dentists and oral surgeons use sutures in the practice of dental medicine, suturing is performed by specialty doctors including eye doctors, and is widely used in veterinary medicine. Sutures are manufactured in a variety of materials, are classified as absorbable or non-absorbable, and are produced in a variety of sizes. Medical professionals select the appropriate type of suture for each procedure and it is imperative that they have access to the preferred suture and have access in a timely fashion. Given the vast array of available sutures, there exists a need for a suture inventory management device that ensures each type of suture is readily available to medical professionals when needed. There further exists a need for such a device to alert staff when stock of a given supply requires replenishment.

### SUMMARY OF DISCLOSURE

The present disclosure relates to a suture inventory management device that dispenses individual units of sutures from a first compartment and stores bulk quantities of the same type of sutures for future use in a second compartment visually and physically connected to the first compartment. The quantities of units of sutures and boxes of sutures in both compartments are visible, thereby providing visual cues to nurses and other staff to restock supply of sutures as needed.

In accordance with aspects and embodiments, a suture storage device is disclosed comprising a housing having two side walls, a bottom, a top, and a first and second open face between the two side walls, the housing having at least one suture storage system positioned within the housing, and the at least one suture storage system including at least one pair of suture storage compartments, the pair of suture storage compartments comprising a bulk suture storage compartment and a suture unit storage compartment. The suture unit storage compartment is positioned above and shares a wall with the bulk suture storage compartment such that the ceiling of the bulk suture storage compartment is the floor of the suture unit storage compartment.

In accordance with embodiments, the suture unit storage compartment comprises a hinged lid and a transparent door, where the lid can be opened to provide access to the interior of the suture unit storage compartment and the transparent door slides in a vertical direction to provide access to the interior the suture unit storage compartment.

In accordance with embodiments, the housing has suture storage systems positioned at the first open face of the housing and at the second open face of the housing. In some embodiments, the device comprises a total of six suture storage systems, with three suture storage systems on each open face of the housing. In other embodiments, the device comprises a total of eight suture storage systems, with four suture storage systems on each open face of the housing. and the device comprises a total of six suture storage systems, with three on each open face of the housing.

In accordance with embodiments, each suture storage system may comprise seven pairs of suture storage compartments and the bulk suture storage compartments of each suture storage system may be enclosed by two transparent hinged doors.

In some embodiments, each pair of suture storage compartments may include a suture-type label and the storage device may include a location label. The suture storage device may further include casters that allow the device to be easily moved.

In accordance with aspects and embodiments, a suture inventory management system is disclosed, the system comprising a suture storage device comprising a plurality of pairs of suture storage compartments, where each pair of suture storage compartments includes a suture unit storage compartment positioned above a bulk suture storage compartment and where each suture unit storage compartment is configured to store a quantity of units of sutures in an interior of the suture unit storage compartment and each bulk suture storage compartment is configured to store a quantity of a boxes of suture containing a plurality of units of sutures in an interior of the bulk suture unit storage compartment. Each pair of suture storage compartments stores the same type of sutures and the quantity of units of sutures and boxes of sutures in each compartment is visible.

In accordance with embodiments, the face of each of the suture unit storage compartments is a transparent door that opens by sliding in a vertical direction and allows access to the units of sutures stored in the unit suture storage compartment. The suture unit storage compartments further comprise a hinged top that opens to allow access to the interior of the compartment.

In accordance with embodiments, one or more of the bulk suture storage compartments is enclosed by a transparent hinged door that opens outward to allow access to the interior of the one or more bulk suture storage compartments.

In accordance with embodiments, units of sutures are loaded into the suture storage device by opening the hinged tops of the suture unit storage compartments and inserting the units of sutures into the suture unit storage compartments, and units of sutures are retrieved from the suture storage device by sliding the transparent doors of the suture unit storage compartments upward to allow access to the units of sutures stored in the suture unit storage compartments.

In accordance with embodiments, the bulk suture storage compartments are restocked with boxes of sutures in response to visual cues received from the bulk suture storage compartments and the suture unit storage compartments are restocked with units of sutures in response to visual cues received from the suture unit storage compartments.

### BRIEF DESCRIPTION OF THE FIGURES

FIG. 1 shows a suture storage device in accordance with aspects and embodiments of the disclosure;

FIG. 2 shows a suture storage system in accordance with aspects and embodiments of the disclosure;

FIG. 3 shows a suture storage system in accordance with aspects and embodiments of the disclosure;

FIG. 4 shows a suture storage system in accordance with aspects and embodiments of the disclosure;

FIG. 5 shows a suture storage system in accordance with aspects and embodiments of the disclosure;

FIG. 6 shows a suture storage system in accordance with aspects and embodiments of the disclosure; and

FIG. 7 shows a flow chart of a suture inventory management system in accordance with aspects and embodiments of the disclosure.

### DETAILED DESCRIPTION

The present disclosure provides a device for the storage of sutures. The sutures of the present disclosure may be used in any medical procedure, including but not limited to human and mammalian surgical procedures and wound management and care. The disclosed device advantageously provides bulk storage and access to individual suture units for use in procedures.

Sutures are generally packaged by unit. A given unit of sutures may include 12 sutures, meaning 12 pieces of medical thread are packaged in the unit. Units are further packaged into boxes containing several units. Storage of sutures is generally by the box, whereas use of sutures in medical procedures is by the unit.

Sutures are classified as either absorbable or non-absorbable depending on whether the body will naturally degrade and absorb the suture material over time. Absorbable sutures are generally used for internal body tissues such that the sutures hold the tissue together long enough to allow healing but disintegrate over time so that they do not leave foreign material in the body or require further medical procedures. Absorbable sutures are made from catgut, as well as newer synthetic materials like polyglycolic acid, polylactic acid, polydioxanone, and caprolactone.

Non-absorbable sutures are typically used for skin wound closure or in internal environments where absorbable sutures would be insufficient. Non-absorbable sutures are made of specialty silk or synthetic materials likes polypropylene, polyester, or nylon, and are sometimes coated with materials to enhance performance. In some procedures, stainless steel wires are used.

Moreover, each suture type and material comes in a variety of sizes depending on intended use. Suture sizes are defined by the United States Pharmacopeia (USP) and have diameters ranging from about 0.01 mm, a USP designation of 11-0, to about 0.8 mm, a USP designation of 6. The actual diameter of thread for a given USP size differs depending on the suture material class.

In accordance with aspects and embodiments, the disclosed device stores boxes of up to 56 different types/sizes of sutures in a first set of compartments and stores corresponding units of those sutures in corresponding unit compartments. In some embodiments, the device is tailored to store a particular amount of suture sizes/types depending on medical needs of the facility in which the device is being used.

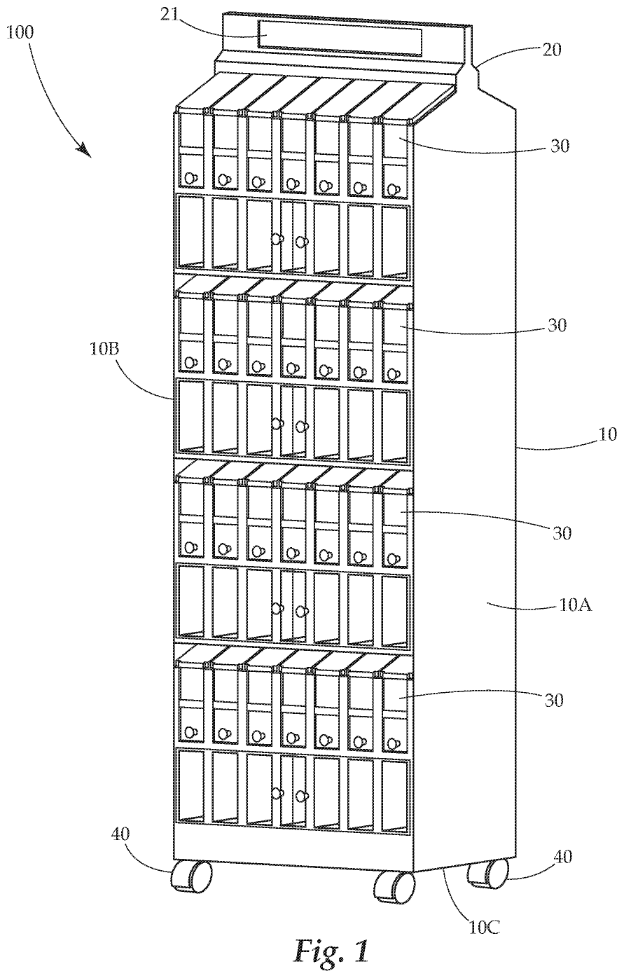

A suture inventory management device in accordance with aspects and embodiments of the disclosure is shown in FIG. 1. Device 100 is in the form of a cart having a housing 10. Housing 10 has side walls 10A and 10B connected by bottom 10C and top piece 20. Top piece 20 may have label 21 that indicates the intended location of cart 100. Cart 100 has 8 identical suture storage systems positioned between side walls 10A and 10B, 4 on each side of cart 100, positioned in parallel above one another. Cart 100 has casters 40 that allow cart 100 to be spun to allow easy access to the other 4 suture storage systems and to allow cart 100 to be easily moved throughout a medical procedure room and elsewhere as needed.

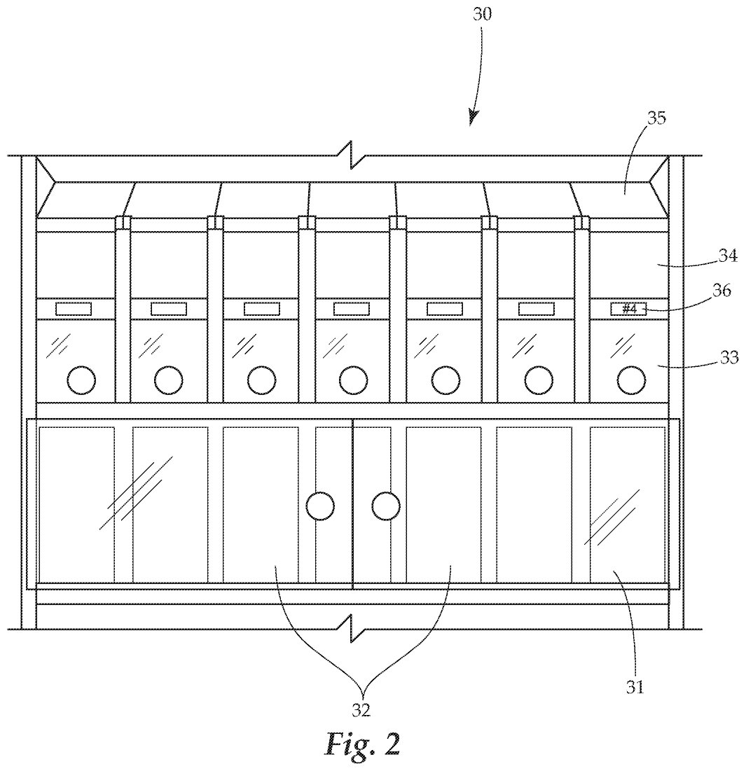

A suture storage system 30 in accordance with aspects and embodiments is shown in FIG. 2. Suture storage system 30 has 7 pairs of suture storage compartments, where each pair of suture storage compartments includes a bulk suture storage compartment 31 and a suture unit storage compartment 33. Suture storage system 30 has 7 bulk suture storage compartments 31 positioned side by side on another. Bulk suture storage compartments 31 are shown as having identical dimensions but may, in some embodiments, have varying widths to accommodate suture boxes of varying sizes. Bulk suture storage compartments 31 are enclosed by transparent doors 32. Doors 32 may be made of any transparent material and may, for example, by made of plexiglass or glass and may further include antimicrobial resistant coatings.

Suture storage system 30 has, positioned above each bulk suture storage compartment 31, and a corresponding suture unit storage compartment 33. The ceiling of each compartment 31 forms the floor of each compartment 33. Suture storage system 30 thus has 7 suture unit storage compartments 33. Each suture unit storage compartment 33 is enclosed by a transparent door 34 and a lid 35. Doors 34 are shown in FIG. 2 as having knobs but may have handles or may simply be pushed to open. Doors 34 may be made of any transparent material and may, for example, by made of plexiglass or glass and may further include antimicrobial resistant coatings. Each pairing of bulk suture storage compartments 31 and suture unit storage compartments 33 may have a label 36 indicating the type of suture contained in the bulk and unit compartments. Doors 34 may latch securely in place to ensure that the sutures contained within compartment 33 remain sterile.

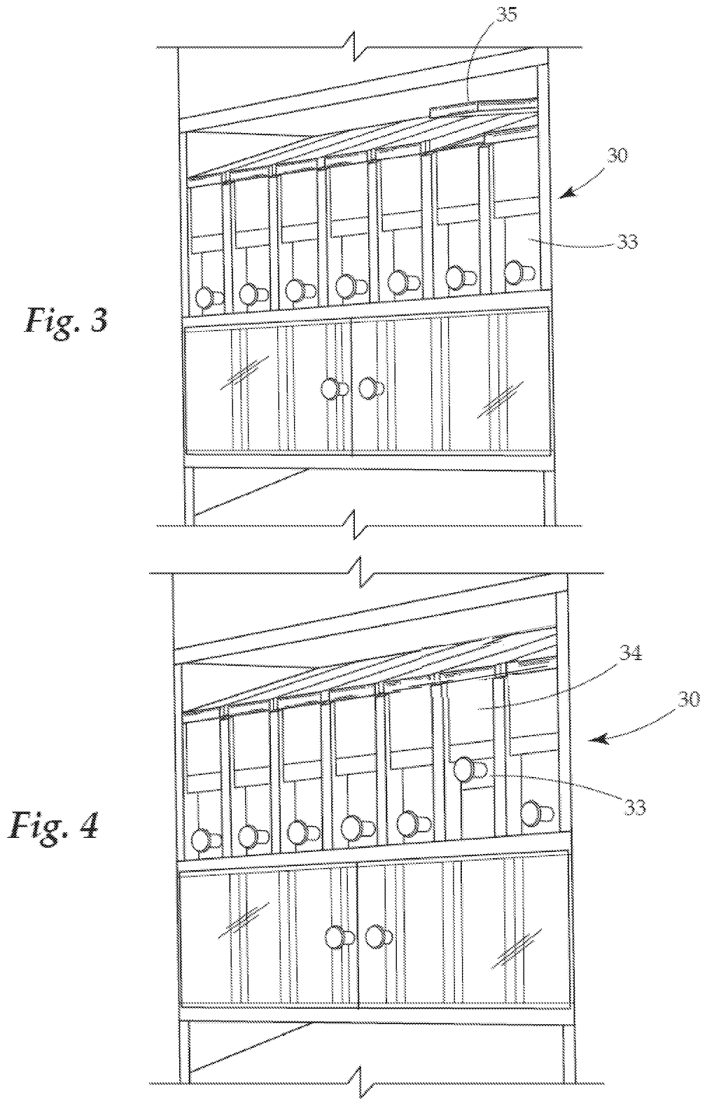

Referring to FIG. 3, units of sutures are loaded into suture unit compartments 33 via the top of suture unit compartment 33. Lid 35 is hinged at the back of compartment 33 and flips upward to provide access to the interior of compartment 33. Lid 35 may hang slightly over the top of compartment 33 creating a lip so that it may easily be flipped upward. Alternatively, lid 35 may have spring mechanism that opens lid 35 when lid 35 is depressed or lid 35 may have a knob that may be used to engage lid 35. Units of sutures are loaded into compartment 33 and lid 35 is then repositioned to close compartment 33. Lid 35 may further include a clip or other locking feature that holds lid 35 in place, ensuring that inventory remains sterile.

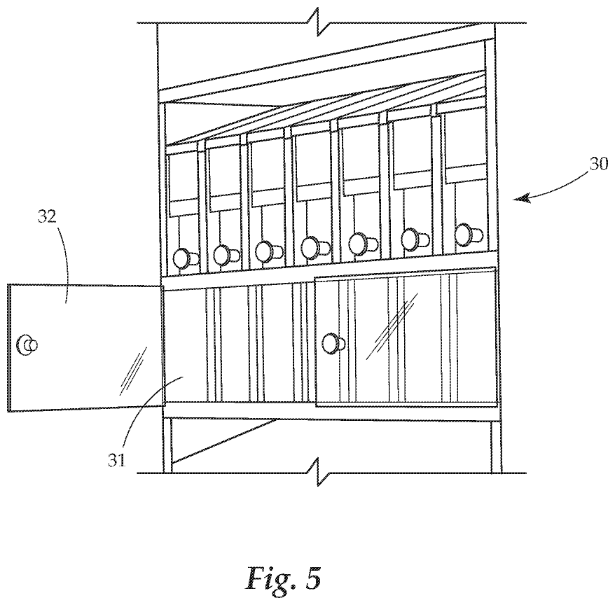

Referring to FIG. 4, when a unit of sutures is required for a procedure, the unit is dispensed via a gravity feed mechanism. Door 34 of suture unit compartment 33 is slid upwards towards top 20 of the device. Door 34 is shown as having a knob in FIG. 4 though door 34 may instead have a handle or other known structure. A unit of sutures may be retrieved from compartment 33 and the next unit of sutures will, by gravity, fall to the floor of compartment 33, positioning it for easy accessibility when another unit of sutures is needed. A second unit of sutures may, for example, be removed, and the process may be repeated or door 34 may be slid downward towards bottom 10C, closing compartment 33. If more units of sutures are removed from compartment 33 than are used for a given procedure, they may be returned to compartment 33 via the opening of lid 35. Turning to FIG. 5, the interior of bulk suture storage compartments 31 are accessed by opening doors 32. Doors 32 are hinged at sides 10A and 10B of housing 10 and open outwardly allowing access to bulk compartments 31. Doors 32 may have a latch that mates with a corresponding feature on the face of one or more walls of the bulk storage compartment 31 to ensure that doors 32 do not unintentionally open. Appropriate latches will be readily ascertained by those of skill in the art and may include magnetic and spring latches.

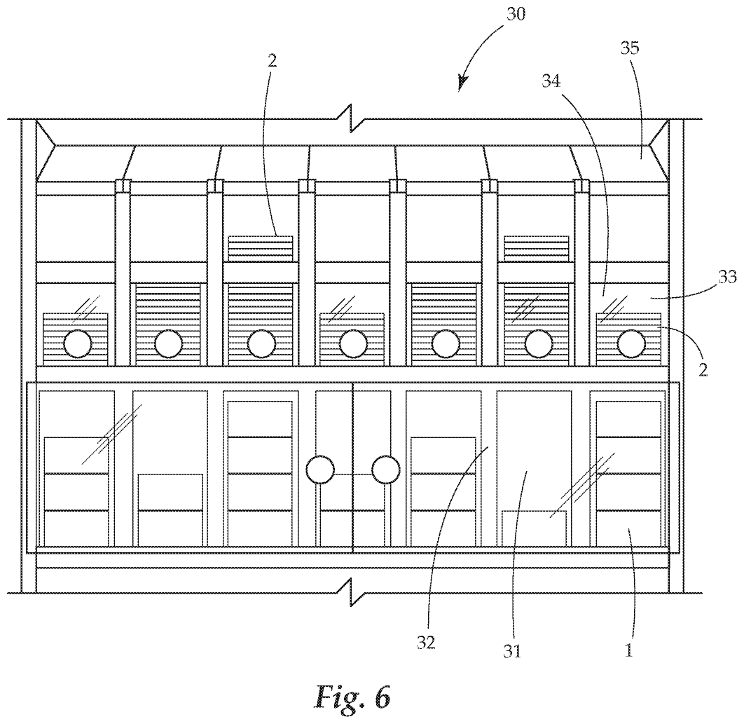

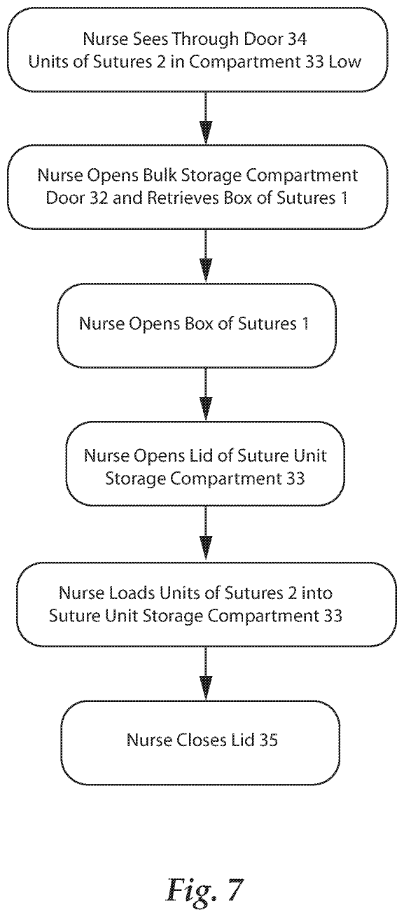

For example, FIG. 6 shows suture storage system 30 having boxes of sutures 1 in bulk storage compartments 31 and units of sutures in suture unit storage compartments 33. In some embodiments, bulk storage compartments 31 may be sized to hold a single box of sutures 1. In alternative embodiments and as shown in FIG. 6, bulk storage compartments 31 may hold a plurality of boxes of sutures 1. Each box of sutures 1 contains a plurality of units of sutures 2. Suture unit compartment 33 may hold the entire quantity of sutures 2 in box 1 or may hold more or less than the quantity of units of sutures 2 in box 1. Turning also to FIG. 7, as units of sutures 2 are used and supply becomes low, the visual appearance of low supply in a given compartment will provide a visual cue to nurses and other staff members to restock inventory. For example, when a nurse of other staff member sees, via a given transparent door 34 of a given suture unit storage compartments 33 that a type of suture in a suture storage unit compartment 33 is in short supply, the nurse will be cued to perform restocking of that compartment 33. The nurse will open the corresponding bulk storage compartment door 32 and retrieve box of sutures 1 and close door 32. The nurse will open box of sutures 1 and open lid 35 to provide access to the interior of suture unit storage compartment 33. The nurse will remove the units of sutures 2 from box of sutures 1 and load units of sutures 2 into suture unit storage compartment 33. The nurse will close lid 35 completing the restocking sequence. Similarly, when a nurse or other staff members sees, via transparent doors 32, that a quantity of boxes of sutures 2 is in short supply, this visual cue will cause restocking of the bulk storage compartment. the supply of boxes of sutures 2 can be restocked from a central storage location via the opening of transparent doors 32.

The transparent nature of the each of doors 32 and 34 ensure that visible cues are provided to alert nurses, doctors, and staff when a quantity of units of sutures or boxes of sutures requires restocking. The gravity feed dispensing of units of sutures from compartment 33 further ensures that the inventory being used has the nearest expiration date and subsequent inventory that is unboxed is loaded on top of the older inventory. This reduces the likelihood of expired inventory. Boxes and units of sutures are also advantageously stored in the original manufacturer's box, ensuring that the products contained therein are correctly labeled and their sterility has not been compromised. The complete enclosure of storage of the units of sutures and boxes of sutures keeps all inventory within Infection Control Guidelines. In the event of a biohazard, only the outside surfaces of storage device may require cleaning. In some embodiments, the device may be made entirely of antimicrobial plastic.

In some embodiments, the number of bulk storage compartments and unit storage compartments may be different than shown in cart 100 to accommodate different applications. In some embodiments, the suture storage device may have suture storage systems on only one face. The same system may alternatively be implemented on a fixed surface, like a wall. The bulk compartments and unit compartments may include color code and may further include sensors to alert nurses and other staff when a given supply of sutures is low. These sensors may cause a light to glow when supply is low, alerting staff to restock the compartment.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A suture storage device comprising:
a housing having two side walls connected by a bottom and a top, the housing having a first open face and a second open face between the two side walls;
at least one suture storage system positioned within the housing, the at least one suture storage system including at least one pair of suture storage compartments, the pair of suture storage compartments comprising a bulk suture storage compartment and a suture unit storage compartment,
where the suture unit storage compartment is positioned above and shares a wall with the bulk suture storage compartment such that the ceiling of the bulk suture storage compartment is the floor of the suture unit storage compartment.

2. The suture storage device of claim 1, wherein the suture unit storage compartment comprises a hinged lid that opens to provide access to an interior of the suture unit storage compartment.

3. The suture storage device of claim 2, wherein the suture unit storage compartment comprises a transparent door.

4. The suture storage device of claim 3, wherein the transparent door slides in a vertical direction to provide access to the interior of the suture unit storage compartment.

5. The suture storage device of claim 4, wherein the housing has suture storage systems positioned at the first open face of the housing and at the second open face of the housing.

6. The suture storage device of claim 5, wherein the device comprises six suture storage systems.

7. The suture storage device of claim 6, wherein the device comprises three suture storage systems positioned at the first open face and three suture storage systems positioned at the second open face.

8. The suture storage device of claim 5, wherein the device comprises eight suture storage systems.

9. The suture storage device of claim 8, wherein the device comprises four suture storage systems positioned at the first open face and four suture storage systems positioned at the second open face.

10. The suture storage device of claim 9, wherein each suture storage system comprises seven pairs of suture storage compartments.

11. The suture storage device of claim 10, wherein the bulk suture storage compartments of each suture storage system are enclosed by two transparent hinged doors.

12. The suture storage device of claim 11, wherein each pair of suture storage compartments has a suture-type label.

13. The suture storage device of claim 12, further comprising a location label.

14. The suture storage device of claim 13, further comprising a plurality of casters affixed to the bottom of the housing.

15. A suture inventory management system comprising:
a suture storage device comprising a plurality of pairs of suture storage compartments, where each pair of suture storage compartments includes a suture unit storage compartment positioned above a bulk suture storage compartment,
where each suture unit storage compartment is configured to store a quantity of units of sutures in an interior of the suture unit storage compartment and each bulk suture storage compartment is configured to store a quantity of a boxes of suture containing a plurality of units of sutures in an interior of the bulk suture unit storage compartment;
wherein each pair of suture storage compartments stores the same type of sutures; and
the quantity of units of sutures in each of the suture unit storage compartments is visible and the quantity of boxes of sutures in each of the bulk suture storage compartment is visible.

16. The suture inventory management system of claim 15, wherein a face of each of the suture unit storage compartments is a transparent door that opens by sliding in a vertical direction and allows access to the units of sutures stored in the unit suture storage compartment.

17. The suture management system of claim 16, wherein each of the suture unit storage compartments further comprise a hinged top that opens to allow access to the interior of the suture unit storage compartment.

18. The suture management system of claim 17, wherein one or more of the bulk suture storage compartments is enclosed by a transparent hinged door that opens outward to allow access to the interior of the one or more bulk suture storage compartments.

19. The suture management system of claim 18, wherein units of sutures are loaded into the suture storage device by opening the hinged tops of the suture unit storage compartments and inserting the units of sutures into the suture unit storage compartments and units of sutures are retrieved from the suture storage device by sliding the transparent doors of the suture unit storage compartments upward to allow access to the units of sutures stored in the suture unit storage compartments.

20. The suture management system of claim 19, wherein the bulk suture storage compartments are restocked with boxes of sutures in response to visual cues received from the bulk suture storage compartments and the suture unit storage compartments are restocked with units of sutures in response to visual cues received from the suture unit storage compartments.
